(19) United States
(12) Patent Application Publication
(10) Pub. No.: US 2012/0070432 A1
(43) Pub. Date: Mar. 22, 2012

(54) TREATMENT OF PANCREATIC CANCER USING A DR5 AGONIST IN COMBINATION WITH GEMCITABINE

(75) Inventors: Jeffrey Scott Wiezorek, Malibu, CA (US); Jonathan David Graves, Seattle, WA (US); Jennifer Joy Kordich, Seattle, WA (US)

(73) Assignee: AMGEN INC., Thousand Oaks, CA (US)

(21) Appl. No.: 13/322,118

(22) PCT Filed: May 27, 2010
(86) PCT No.: PCT/US10/36419

§ 371 Date: Nov. 22, 2011

Related U.S. Application Data
(60) Provisional application No. 61/182,034, filed on May 28, 2009.
(60) Provisional application No. 61/345,015, filed on May 14, 2010.

(51) Int. Cl. A61K 39/395; A61P 35/00; C12Q 1/68

(52) U.S. Cl. 424/133.1; 424/152.1; 424/142.1; 424/172.1; 435/6.11

(57) ABSTRACT

Methods and compositions for treatment of exocrine pancreatic cancer in a human patient comprising administering a therapeutically effective amount of a DR5 agonist and gemcitabine. Methods and compositions for treating a patient by identifying the alleleic variant of FcγRIIIA.

### CROSS REFERENCE TO RELATED APPLICATIONS

This application claims the benefit under 35 U.S.C. 119(e) of U.S. patent application No. 61/182,034 filed May 28, 2009 and U.S. patent application 61/345,015 filed May 14, 2010 which are incorporated herein by reference.

### FIELD OF THE INVENTION

The present invention relates to a method of inhibiting the growth of pancreatic cancer in a human patient by administering a therapeutically effective amount of a DR5 agonist in combination with gemcitabine.

### BACKGROUND OF THE INVENTION

The interaction between DR5 (alternatively referred to as TRAIL Receptor-2, TR-2, or Apo2) and its ligand, TRAIL (TNF-Receptor Apoptosis Inducing Ligand), plays a role in the induction of apoptosis of cancer cells. TRAIL, also known as Apo2 ligand, is a homotrimeric ligand that interacts with four members of the TNF-receptor superfamily (TRAIL receptors (“TR”) 1 to 4), as well as with the related, soluble, opsteoprotegerin (“OPG”) receptor. Binding of TRAIL to DR4 (TRAIL Receptor-1; TR-1) or DR5 at the surface of a sensitive cancer cell triggers an apoptotic cascade. After initial binding of TRAIL to DR5 or DR4, intracellular proteins are recruited to the intracellular death domain of the receptor, forming a signaling complex. Certain intracellular caspases are recruited to the complex, where they autoactivate and in turn activate additional caspases and the intracellular apoptosis cascade leading to cell death. In addition to TRAIL, other agonists of DR4 and/or DR5 can likewise induce apoptosis in certain cancer cells.

Approximately 100,000 individuals are diagnosed each year with pancreatic cancer in the U.S. and Europe. Prognosis of patients is poor with a survival rate five years post-diagnosis of less than 5%. The pancreas contains two different types of parenchymal tissue: exocrine and endocrine that form different tumor types. Approximately 95% of exocrine pancreatic cancers are adenocarcinomas. The remaining 5% include adenosquamous are far more common than endocrine pancreatic cancers which make up about 1% of total cases.

An effective treatment to shrink, cease growth of, and/or otherwise slow progression of pancreatic cancer, particularly adenocarcinoma, is needed. Accordingly, it is an object of the present invention to provide a method of inhibiting the growth of pancreatic cancer by administration of a therapeutically effective dose of a DR5 agonist in combination with the chemotherapeutic agent gemcitabine. It is also an object of the present invention to identify a predictive biomarker of clinical efficacy in the treatment of adenocarcinoma of the pancreas by the combination therapy of the present invention.

### SUMMARY OF THE INVENTION

The present invention is directed in part to a method of inhibiting the progression of exocrine pancreatic cancer in a human patient by administering a therapeutically effective amount of a DR5 agonist in combination with gemcitabine. The DR5 agonist of the present invention can be an antibody, apo2L/TRAIL, avimer, Fc-peptide fusion protein (such as a peptibody), or a small molecule DR5 agonist. Compositions comprising a DR5 agonist and gemcitabine for use in the methods of the invention are also provided. In another aspect, the invention is directed to methods, assays, and assay kits for identifying human patients with adenocarinoma of the pancreas who are homozygous or heterozygous for the V158 polymorphism of FcγRIIIA (CD16) and thus have a statistically increased likelihood of obtaining a clinical benefit by treatment with a DR5 agonist of the present invention (comprising an IgG1 Fc) in combination with gemcitabine. Also provided are DR5 agonists comprising a modified IgG1 Fc to improve the clinical benefit obtained from the combination therapy for human patients homozygous for the F158 polymorphism of FcγRIIIA.

### DETAILED DESCRIPTION OF INVENTION

The present invention relates to compositions and methods for inhibiting progression of exocrine pancreatic cancer in a human patient by administering a therapeutically effective amount of a DR5 agonist in combination with gemcitabine.

The section headings are used herein for organizational purposes only, and are not to be construed as in any way limiting the subject matter described. The disclosure of all patents, patent applications, and other documents cited herein are hereby expressly incorporated utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

Definitions

The terms used throughout this specification are defined as follows, unless otherwise limited in specific instances.

The term “afucosylation” or “afucosylated” in the context of an Fc refers to a substantial lack of a fucosyl group covalently attached, directly or indirectly, to residue 297 of the human IgG1 Fc numbered according to the EU index (Kabat et al., Sequences of Proteins of Immunological Interest, 5th Ed. Public Health Service, National Institutes of Health, Bethesda, Md. (1991)), or the corresponding residue in non-IgG1 or non-human IgG1 immunoglobulins. Thus, in a composition comprising a plurality of afucosylated Fc-polypeptides at least 80% of the Fc-polypeptides will be not be fucosylated, directly or indirectly (e.g., via intervening sugars) at residue 297 of the Fc, and in some embodiments at least 80%, 85%, 90%, 95%, or 99% will not be fucosylated, directly or indirectly at residue 297 of the Fc.

The term “DR5” or TRAIL-R” or “Apo-2” or “TR-2” or “TRAIL Receptor-2” refer to the 440 amino acid polypeptide set forth in SEQ ID NO: 2 of U.S. Pat. No. 7,528,239 as well as related native (i.e., wild-type) human polypeptides such as allelic variants or splice variants such as, but not limited to, the 411 amino acid isoforms set forth in SEQ ID NO: 1 in U.S. Pat. No. 6,342,369, and at SEQ ID NO: 2 of U.S. Pat. No. 6,743,625 (each patent incorporated herein by reference), including mature forms of the polypeptide (i.e., lacking a leader sequence).

The term “DR5 agonist” refers to a composition that specifically binds to cells expressing native human DR5 and triggers an apoptotic cascade resulting in a statistically significant increase in cell death (i.e., apoptosis) as measured in at least one DR5 agonist sensitive cell line (including, but not limited to, the human colon carcinoma cell line Colo 205, or the human lung carcinoma cell line H2122). In certain embodiments, the DR5 agonist is an antibody, peptibody, avimer (Nature Biotechnology 23:1556-1561 (2005)), or human TRAIL ligand (see, U.S. Pat. Nos. 6,284,236; 6,998,116, both of which are incorporated herein by “small molecule”) DR5 agonist (e.g., U.S. Ser. No. 11/866,162 (Srivastava et al.).

The term “antibody” includes reference to isolated forms of both glycosylated and non-glycosylated immunoglobulins of any isotype or subclass, including any combination of: 1) human (e.g., CDR-grafted), humanized, and chimeric antibodies, 2) monospecific (e.g., DR5) or multi-specific antibodies (e.g., DR4 and DR5), and 3) monoclonal, polyclonal, or single chain (scFv) antibodies, irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody (e.g., from a transfectoma), (c) antibodies isolated from a recombinant, combinatorial antibody library, and (d) antibodies prepared, expressed, created or isolated by any other means that involve splicing of immunoglobulin gene sequences to other DNA sequences. In some embodiments the antibodies of the present invention are monoclonal antibodies, such as humanized or fully-human monoclonal antibodies. Typically, antibodies of the present invention will be IgG1 or IgG2 subclass antibodies. The antibody may bind DR5 with a Kd of less than about 10 nM, 5 nM, 1 nM, or 500 pM.

The terms “derivation” or “derivatives” refer to modification of a DR5 agonist (such as an antibody) and/or gemcitabine by covalently linking it, directly or indirectly, so as to modify such characteristics as half-life, bioavailability, immunogenicity, solubility, or hypersensitivity properties, while retaining its therapeutic benefit. Derivatives can be made by glycosylation, pegylation, and lipidation, or by protein conjugation. Exemplary derivitizing agents include an Fc domain as well as a linear polymer (e.g., polyethylene glycol (PEG), polylysine, dextran, etc.); a branched-chain polymer (See, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid or liposome; a cholesterol group (such as a steroid); a carbohydrate or oligosaccharide.

The terms “effective amount” or “therapeutically effective amount” refer to an amount of a DR5 agonist that when administered to a human patient for treatment of pancreatic cancer in combination with an amount of gemcitabine typically used in chemotherapeutic treatment of adenocarcinoma (pancreatic cancer) (e.g., about 1000 mg/m²), yields a statistically significant inhibition of pancreatic cancer progression relative to the same dosage of pancreatic cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in survival relative to treatment with gemcitabine alone.

The term “Fc” in an antibody or peptibody of the present invention is typically fully human Fc, and may be any of the immunoglobulins, although IgG1 and IgG2 are typical. However, Fc molecules that are partially human, or obtained from non-human species are also included herein.

The term “Fc-peptide fusion” refers to a peptide that specifically binds to and agonizes DR5 when covalently bonded, directly or indirectly, to an Fc. Exemplary Fc-peptide fusion include peptibodies (WO 2000/24782, incorporated herein by reference). For example, an Fc-peptide fusion may be an Fc-human TRAIL ligand fusion.

The term “high-affinity” in the context of a DR5 agonist comprising an Fc means that the Fc specifically binds to human FCGR3A expressed by a native cell (e.g., a human NK cell) that is homozygous for the F158 allele with at least the same affinity as at least one of: an identical but afucosylated DR5 agonist (e.g., an antibody), or an identical DR5 agonist but comprising a modification to increase FCGR3A affinity at residue 332 of the Fc (per EU index of Kabat; see, U.S. Pat. No. 7,317,091 and/or U.S. Pat. No. 7,662,925) such as an isoleucine to glutamic acid substitution. Generally a high-affinity DR5 agonist specifically binds to human FCGR3A with at least the same affinity as a native fucosylated Fc of a DR5 agonist binds to human FCGR3A expressed by a native cell that is homozygous for the V158 allele. Means to measure binding affinity are known in the art and include but are not limited to competition assays such as an AlphaLISA™ (Perkin Elmer, Waltham, Mass. USA) ELISA assay. See, Poulsen, J., et al. 2007. J. Biomol Screen. 12:240, Cauchon, E., et al. 2009. Anal Biochem.

The term “host cell” refers to a cell that can be used to express a nucleic acid, e.g., a nucleic acid of the present invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media (see Rasmussen et al., et al., Proc. Natl. Acad. Sci. USA 77: 4216-4220, 1980).

The term “human antibody” or “fully human antibody” refers to an antibody in which both the constant regions and the framework consist of fully or substantially human sequences such that the human antibody typically elicits substantially no immunogenic reaction against itself when administered to a human and, preferably, elicits no detectable immunogenic response. Thus, the defined terms contemplate minor amino acid modifications (often no more than 1, 2, 3, or 4 amino acid substitutions, additions, or deletions) made relative to a native human antibody sequence to allow, for example, for improved formulation or manufacturability (e.g., removal of unpaired cysteine residues).

The term “humanized antibody” refers to an isolated antibody in which substantially all of the constant region is derived from or corresponds to human immunoglobulins, while all or part of one or more variable regions is derived from another species, for example a mouse.

The term “isolated” refers to a compound that: (1) is substantially purified (e.g., at least 60%, 70%, 80%, or 90%) away from cellular components with which it is admixed in its expressed state such that it is the predominant species present, (2) is conjugated to a polypeptide or other moiety to which it is not linked in nature, (3) does not occur in nature as part of a larger polypeptide sequence, (4) is combined with other chemical or biological agents having different specificities in a well-defined composition, or (5) comprises a human engineered sequence not otherwise found in nature.

The terms “monoclonal antibody” or “monoclonal antibody composition” refers to a preparation of isolated antibody molecules of single molecular composition, typically encoded by the same nucleic acid molecule. A monoclonal antibody composition displays a single binding specificity and affinity for a particular epitope. In certain embodiments, monoclonal antibodies are produced by a single hybridoma or other cell line (e.g., a transfectoma), or by a transgenic mammal. The term “monoclonal” is not limited to any particular method for making an antibody.

The term “naturally occurring” or “native” when used in connection with biological materials such as nucleic acid molecules, polypeptides, host cells, and the like, refers to those which are found in nature and not modified by a human being.

The terms, “nucleic acid” and “polynucleotide” refer to a deoxyribonucleotide or ribonucleotide polymer, or chimeras thereof, and unless otherwise limited, encompasses the linked” to a regulatory sequence if the regulatory sequence affects the expression (e.g., the level, timing, or location of expression) of the nucleic sequence. A “regulatory sequence” is a nucleic acid that affects the expression (e.g., the level, timing, or location of expression) of a second nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., Nucleic Acids Res. 23: 3605-3606, 1995.

The terms “peptide,” “polypeptide” and “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “peptibody” refers to a specific binding agent that is a molecule comprising an antibody Fc domain attached to at least one peptide. The production of peptibodies is generally described in PCT publication WO 00/24782, published May 4, 2000, incorporated herein by reference. Exemplary peptides may be generated by any of the methods set forth herein, such as carried in a peptide library (e.g., a phage display library), generated by chemical synthesis, derived by digestion of proteins, or generated using recombinant DNA techniques.

The terms “peptibody fragment” or “antibody fragment” refers to a peptide or polypeptide of an antibody or peptibody specific binding agent which comprises less than a complete intact antibody or peptibody but retains the ability to specifically bind to its target molecule (i.e., human DR5). Exemplary fragments includes F(ab) or F(ab′)2 fragments. Such a fragment may arise, for example, from a truncation at the amino terminus, a truncation at the carboxy-terminus, and/or an internal deletion of a residue(s) from the amino acid sequence. Fragments may result from alternative RNA splicing or from in vivo or in vitro protease activity. Such fragments may also be constructed by chemical peptide synthesis methods, or by modifying a polynucleotide encoding an antibody or peptibody. interchangeably throughout and include DNA molecules (e.g., cDNA or genomic DNA), RNA molecules (e.g., mRNA), and hybrids thereof The nucleic acid molecule can be single-stranded or double-stranded.

The term “specifically binds” refers to the ability of a DR5 agonist of the present invention, under specific binding conditions, to bind to a cell surface human DR5 molecule such that its affinity is at least 10 times as great, but optionally 50 times as great, 100, 250 or 500 times as great, or even at least 1000 times as great as the average affinity of the same binding agent to a collection of random peptides or polypeptides of sufficient statistical size. A specific binding agent need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid representation of unique non-targets (e.g., random polypeptides). Thus, a DR5 agonist of the invention may specifically bind to more than one distinct species of target molecule, such as specifically binding to both DR5 and DR4. Solid-phase ELISA immunoassays can be used to determine specific binding. Generally, specific binding proceeds with an association constant of at least about 1×10⁷ M⁻¹, and often at least 1×10⁸ M⁻¹, 1×10⁹ M⁻¹, or, 1×10¹⁰ M⁻¹.

The term “vector” refers to a nucleic acid used in the introduction of a polynucleotide of the present invention into a host cell. Vectors are often replicons. Expression vectors permit transcription of a nucleic acid inserted therein when present in a suitable host cell or under suitable in vitro conditions.

Combination Therapy for Treatment of Pancreatic Cancer

The present invention is directed to a method of treating exocrine pancreatic cancer (adenocarcinoma of the pancreas) in a human patient so as to inhibit, halt, or reverse progression of the tumor, or otherwise result in a statistically significant increase in progression-free survival (i.e., the length of time during and after treatment in which a patient is living with pancreatic cancer that does not get worse), or overall survival (also called “survival rate”; i.e., the percentage of people in a study or treatment group who are alive for a certain period of time after they were diagnosed with or treated for pancreatic cancer) relative to therapeutically effective amount of a DR5 agonist in combination with gemcitabine (GEMZAR). In some embodiments, the DR5 agonist (e.g., an antibody) is administered to the patient at from 0.3 to 30 mg/kg of patient body weight, often at from 2 to 20 mg/kg, or 3 to 15 mg/kg. Gemcitabine is administered in a dose ranging from 250 to 2500 mg/m², more typically 500 to 1250 mg/m², often at approximately 1000 mg/m². The combination is typically administered until disease progression or the point of maximum clinical benefit as determined by the physician.

The DR5 agonist of the present invention specifically binds to and agonizes DR5 thereby activating the apoptotic pathway leading to cell death (apoptosis) in sensitive cancer cells. In some embodiments, the DR5 agonist (e.g., an antibody) also specifically binds to DR4 but does not induce apoptosis via both the DR4 and DR5 receptors in a particular cell type. In other embodiments, the DR5 agonist of the invention specifically binds to and agonizes the DR4 receptor. Thus, a dual DR5 and DR4 agonist of the invention can agonize the same, different, or overlapping populations of cancer cells. In some embodiments the DR5 agonist does not specifically bind to (i.e., does not cross-react) and/or agonize DR4.

In the method of the present invention, a therapeutically effective amount of a DR5 agonist is administered in combination with gemcitabine, a chemotherapeutic agent commercially available to the clinician (GEMZAR, Eli Lilly). Standard dosages and methods of administrations can be used, for example per the Food and Drug Administration (FDA) label. In certain embodiments gemcitabine is administered at approximately 1000 mg/meter² (square meter of patient surface area) in combination with the DR5 agonist antibody, conatumumab, at dosages from about 0.3 mg/kg to about 30 mg/kg, typically about 10 mg/kg.

Gemcitabine of the present invention can be administered prior to and/or subsequent to (collectively, “sequential treatment”), and/or simultaneously with (“concurrent treatment”) a specific binding agent of the present invention. Sequential treatment (such as pretreatment, post-treatment, or overlapping treatment) of the combination, also includes regimens in which the drugs are alternated, or wherein one component is administered long-term and the other(s) are administered intermittently. Components of the combination may be administered in the same or in separate compositions, and by the same or different routes of administration.

The combination therapy of the present invention can be administered to a patient having adenocarcinoma of the pancreas of stage I, II, III, or IV, per the staging criteria established by the American Joint Committee on Cancer (AJCC) using the TNM (Tumor, stage III, or at stage IV.

Gemcitabine may be given as a drip (infusion) through a cannula inserted into a vein (IV), through a central line, which is inserted under the skin into a vein near the collarbone, or a peripherally inserted central catheter (PICC) line. The dose is often administered in a fixed-time such as 30 minutes. Alternatively, the dose can be administered at a fixed rate (e.g., 10 mg/m²/minute). In one embodiment, gemcitabine dosing is administered IV at 1000 mg/m2 every week for days 1, 8, and 15 of a 28-day cycle. In an alternative embodiment, gemcitabine is administered seven weeks in a row, followed by one week off, then 3 out of 4 weeks for subsequent doses.

The DR5 agonist is administered at doses and rates readily determined by those of ordinary skill in the art. In some embodiments, the DR5 agonist is an antibody (e.g., conatumumab) administered intravenously on days 1 and 15 of a 28-day cycle. In some embodiments, 28-day cycle for the DR5 agonist and gemcitabine synchronized such that the DR5 agonist and gemcitabine are both given on days 1 and 15 of the 28-day cycle. In some embodiments, the DR5 agonist antibody, such as conatumumab, is administered to the human patient at about 1 mg/kg, 2 mg/kg, 3 mg/kg, 5 mg/kg, 7 mg/kg, 10 mg/kg, 12 mg/kg, 15 mg/kg, 20 mg/kg, 25 mg/kg, or 30 mg/kg of patient bodyweight.

DR5 Agonists

Specific DR5 agonists of the present invention are known in the art or may be prepared using methods known in the art. Exemplary DR5 agonists are taught and disclosed in, e.g., U.S. Pat. No. 7,521,048 (Gliniak et al.); U.S. Pat. Nos. 6,284,236 (Wiley et al.); 6,998,116 (Ashkenazi et al.); PCT WO 2006/083971 (Adams); PCT WO 2008/004760 (Kim et al.); PCT WO 2003/037913 (Zhou et al.); and, U.S. Ser. No. 11/866,162 (Srivastava et al.), all of which are incorporated herein by reference. In a specific embodiment, the DR5 agonist is conatumumab (AMG 655), CAS Registry Number 896731-82-1, Antibody “O” of U.S. Pat. No. 7,521,048, incorporated herein by reference. Other exemplary DR5 agonists included within the scope of the present invention include: lexatumumab (Human Genome Sciences), CS-1008 (Daiichi Sankyo), LBY-135 (Novartis), and apomab (Genentech).

The isolated DR5 agonist antibodies of the present invention may be isolated polyclonal or isolated monoclonal (mAbs). The isolated polyclonal or monoclonal antibodies can be chimeric, humanized, fully human, single chain, bi-specific, as well as antigen-binding monoclonal antibodies (e.g., conatumumab).

Monoclonal antibodies specifically binding to DR5 can be produced using, for example but without limitation, the traditional “hybridoma” method or the newer “phage display” technique. For example, monoclonal antibodies of the invention may be made by the hybridoma method as described in Kohler et al., Nature 256:495 [1975]; the human B-cell hybridoma technique [Kosbor et al., Immunol Today 4:72 (1983); Cote et al., Proc Natl Acad Sci (USA) 80: 2026-2030 (1983); Brodeur et al., Monoclonal Antibody Production Techniques and Applications, pp. 51-63, Marcel Dekker, Inc., New York, (1987)] and the EBV-hybridoma technique [Cole et al., Monoclonal Antibodies and Cancer Therapy, Alan R Liss Inc, New York N.Y., pp 77-96, (1985)].

The phage display technique may also be used to generate monoclonal antibodies. Preferably, this technique is used to produce fully human monoclonal antibodies in which a polynucleotide encoding a single Fab or Fv antibody fragment is expressed on the surface of a phage particle. [Hoogenboom et al., J Mol Biol 227: 381 (1991); Marks et al., J Mol Biol 222: 581 (1991); see also U.S. Pat. No. 5,885,793)]. Each phage can be “screened” using standard binding and cell-based assays to identify those antibody fragments having affinity for, and agonization of, DR5.

Once polynucleotide sequences are identified which encode each chain of the full length monoclonal antibody or the Fab or Fv fragment(s) of the invention, host cells, either eukaryotic or prokaryotic, may be used to express the monoclonal antibody polynucleotides using recombinant techniques well known and routinely practiced in the art.

In another embodiment of the present invention, a monoclonal or polyclonal antibody or fragment thereof that is derived from other than a human species may be “humanized” or “chimerized”. Methods for humanizing non-human antibodies are well known in the art. (see U.S. Pat. Nos. 5,859,205, 5,585,089, and 5,693,762). Humanization is performed, for example, using methods described in the art [Jones et al., Nature 321: 522-525 (1986); Riechmann et al., Nature, 332: 323-327 (1988); Verhoeyen et al., Science 239:153 4-1536 (1988)] by substituting at least a portion of, for example a rodent, complementarity-determining region (CDRs) for the corresponding regions of a human antibody.

Alternatively, transgenic animals (e.g., mice) that are capable of producing a repertoire of human antibodies in the absence of endogenous immunoglobulin production can be used to generate such antibodies. This can be accomplished by immunization of the animal with a DR5 antigen or fragments thereof (e.g., the DR5 extracellular domain). Such Natl Acad Sci (USA), 90: 2551-2555 (1993); Jakobovits et al., Nature 362: 255-258 (1993); Bruggermann et al., Year in Immuno, 7: 33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, that are those having less than the full complement of these modifications, are then crossbred to obtain an animal having all of the desired immune system modifications. When administered an immunogen, these transgenic animals are capable of producing antibodies with human variable regions, including human (rather than e.g., murine) amino acid sequences, that are immuno-specific for the desired antigens. See PCT application Nos., PCT/US96/05928 and PCT/US93/06926. Additional methods are described in U.S. Pat. No. 5,545,807, PCT application Nos. PCT/US91/245, PCT/GB89/01207, and in EP 546073B1 and EP 546073A1. Human antibodies may also be produced by the expression of recombinant DNA in host cells or by expression in hybridoma cells as described herein.

Large-scale production of chimeric, humanized, CDR-grafted, and fully human antibodies, or antigen-binding fragments thereof, are typically produced by recombinant methods. Polynucleotide molecule(s) encoding the heavy and light chains of each antibody or antigen-binding fragments thereof, can be introduced into host cells and expressed using materials and procedures described herein. In a particular embodiment, the antibodies are produced in mammalian host cells, such as CHO cells.

DR5 Agonist Pharmaceutical Formulation

The pharmaceutical composition comprising the DR5 agonists of the present invention may contain formulation materials for modifying, maintaining or preserving, for example, the pH, osmolarity, viscosity, clarity, color, isotonicity, odor, sterility, stability, rate of dissolution or release, adsorption, or penetration of the composition.

The primary vehicle or carrier in a pharmaceutical composition may be either aqueous or non-aqueous in nature. For example, a suitable vehicle or carrier may be water for injection or physiological saline, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The formulation components are present in concentrations that are acceptable to the site of administration. For example, buffers are used to maintain the composition at physiological pH or at slightly lower pH, typically within a pH range of from about 5 to about 8. A particularly suitable vehicle for parenteral administration is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provide for the controlled or sustained release of the product which may then be delivered via a depot injection.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919; EP 58,481), copolymers of L-glutamic acid and gamma ethyl-L-glutamate [Sidman et al., Biopolymers, 22:547-556 (1983)], poly (2-hydroxyethyl-methacrylate) [Langer et al., J. Biomed. Mater. Res., 15:167-277, (1981)] and [Langer et al., Chem. Tech., 12:98-105(1982)], ethylene vinyl acetate (Langer et al., supra) or poly-D(-)-3-hydroxybutyric acid (EP 133,988). Sustained-release compositions also include liposomes, which can be prepared by any of several methods known in the art. See e.g., EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 50 mg/kg or more, depending on the factors mentioned above. In other embodiments, the dosage may range from 1 mg/kg, 2, 3, 5, 10, 15, up to about 30 mg/kg.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

FCGR3A Polymorphism

A bi-allelic polymorphism of the human IgG receptor FcγRIIIA (CD16) (alternatively, “FCGR3A”) can be distinguished by virtue of the presence of the amino acid valine (V) or phenylalanine (F) at the locus identified at the publicly accessible National Center for Biotechnology Information's (NCBI) Single Nucleotide Polymorphism (SNP) database at cluster report rs396991. These two alleleic forms are commonly referred to in the literature and herein as “valine158” or “V158” for the polymorphism having the residue valine at the rs396991 SNP locus of human FcγRIIIA, and “phenylalanine158” or “F158” for the polymorphism having the residue phenylalanine at the rs396991 SNP locus of human FcγRIIIA. See also, Leppers-van de Straat et al., J. Immunological Methods, 242: 127-132 (2000) and Ravetch and Perussia, J. Exp. Med., 170:481-497 (1989).

The present invention provides a method of identifying a human patient (or patients) having adenocarcinoma of the pancreas who are more likely to obtain a clinical benefit from treatment with the combination therapy of the present invention (i.e., DRS agonist and gemcitabine) as evidenced by a statistically significant increased response in progression-free survival and/or overall survival. Such patients are heterozygous (F158/V158) or, even more preferably, homozygous (V158/V158) for the V158 polymorphism of FcγRIIIA. Patients can be stratified on the basis of this allelic difference and those identified as having one or two combination therapy herein disclosed. Identifying a patient having a V158 and F158 polymorphism can be achieved employing analytical methods known to those of skill in the art such as PCR based methods (Leppers-van de Straat et al., J. Immunological Methods, 242: 127-132 (2000)). Conveniently, a clinician can identify such patients using the services of third party laboratories to carry out such methods. Kits for identifying patients having 0, 1, or 2 copies of the V158 or F158 allele of FcγRIIIA in cancer patients diagnosed of having adenocarcinoma of the pancreas are also within the scope of the present invention. Such kits can optionally contain written instructions identifying the allelic forms of patients who are more likely to respond to the combination therapy (i.e., V158N158 and F158N158 patients).

High Affinity DR5 Agonists

The present invention provides a DR5 agonist which, when comprising an Fc (e.g., antibodies or Fc-fusion peptides), can be made (e.g., constructed or modified) to substantially increase binding affinity to human FCGR3A and yield a high-affinity DR5 agonist. In one embodiment afucosylated DR5 agonists are provided. Reducing or eliminating the extent of IgG1 fucosylation of such DR5 agonists can be used to improve the clinical benefit received from the combination therapy of the present invention relative to an unmodified form of the Fc (i.e., native fucosylation levels), particularly for patients homozygous for F158 of FCGR3A. The Fc of such high-affinity DR5 agonists is generally an IgG1 Fc and typically fully-human in its primary sequence although minor modifications can be made to allow, for example, for improved formulation or manufacturability while FCGR3A binding is not significantly diminished. While such afucosylated DR5 agonists can be administered in a therapeutically effective amount in the combination therapy of the present invention in patients homozygous for F158 polymorphism to improve clinical benefit relative to a control fucosylated Fc of a DR5 agonist, such afucosylated Fc-containing DR5 agonists also have an advantage in that they can also be administered in therapeutically effective amounts to patients heterozygous or homozygous for the V158 allele to substantially maintain or even improve the clinical benefit relative to a control fucosylated Fc of a DR5 agonist. Thus, the present invention provides afucosylated (above 98% or above 99% fucose-free Fc and generally at least 60%, 70%, 80%, 90%, or 95% fucose-free Fc) DR5 agonists compositions for use in the combination therapy of the present invention for substantially all patients regardless of the FCGR3A genotype at SNP locus rs396991. Methods of creating afucosylated (e.g., antibodies) or Fc-fusion peptides are known in the art and include, but are not limited to, recombinant expression using enzymatic or host cells missing the gene for fucosyl transferase (i.e., knock-outs), or defucosylating the Fc by in vitro chemical methods. See, e.g., U.S. Pat. No. 7,317,091 and, U.S. Pat. No. 6,946,292, both incorporated herein by reference.

In some embodiments, the present invention provides a DR5 agonist which, when comprising an Fc, comprises an amino acid substitution as described in U.S. Pat. No. 7,317,091 (incorporated herein by reference) to yield a high-affinity agonist with increased affinity to human FCGR3A. Such modifications to create a high-affinity DR5 agonist can be used to improve the clinical benefit received from the combination therapy of the present invention relative to an unmodified form of Fc (i.e., native Fc) of the DR5 agonist in patients homozygous or heterozygous for F158 of FCGR3A. In some embodiments, the Fc is a human IgG1 Fc. In some embodiments, the DR5 agonist comprising the Fc is an antibody, such as a fully-human monoclonal antibody. In some embodiments, the amino acid residue to be substituted is at least one of residues: 230, 233, 234, 235, 239, 240, 243, 264, 266, 272, 274, 275, 276, 278, 302, 318, 324, 325, 326, 328, 330, 332, and 335, wherein the numbering of the residues in the Fc region is that of the EU index as in Kabat. In some embodiments, the Fc comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V3021, E318R, S324D, S324I, S324V, N325T, K326I, K326T, L328M, L328I, L328Q, L328D, L328V, L328T, A330Y, A330L, A330I, I332D, 1332E, I332N, I332Q, T335D, T335R, and T335Y wherein the letter preceding the number represents in one-letter amino acid code the substitution residue, the number indicates the residue of the Fc numbered per the EU index as in Kabat and the letter following the number indicates the native residue. In some embodiments, the Fc of a DR5 agonist of the present invention (that comprises an Fc) comprises both an afucosylated Fc and an amino acid substituted Fc as described above. In some embodiments, the Fc of the Fc-polypeptide comprises 1, 2, 3, 4, 5, 6, 7, 8, 9, or 10 of the substitutions to increase affinity to FCGR3A.

The above listings are by way of example only, and do not preclude the use of other compounds or treatments which can be used concurrently with the compounds described herein that are known by those skilled in the art or that could be arrived at by those skilled in the art using the guidelines set forth in this specification.

Example 1 describes the treatment of patients with advanced solid tumors with a DR5 agonist as a monotherapy as reported by LoRusso et al., Journal of Clinical Oncology, 2007 ASCO Annual Meeting Proceedings Part I. Vol 25, No. 18S (June 20 Supplement), 2007: 3534 (incorporated herein by reference).

Conatumumab (AMG 655) is a fully human monoclonal agonist antibody that binds human TRAIL receptor 2 (TR-2/DR5), activates caspases, and induces apoptosis in sensitive tumor cells. The primary objectives of this ongoing first-in-human study were to assess the safety, tolerability, and pharmacokinetics (PK) of AMG 655 in patients with advanced solid tumors.

Three to nine patients were enrolled into 1 of 5 sequential dose cohorts (0.3, 1, 3, 10, or 20 mg/kg) of conatumumab (AMG 655) administered intravenously Q2W. No AMG 655 was administered on day 43 to allow assessment of terminal PK parameters. RECIST and FDG-PET were analyzed by central radiology. Patients remained on study until tumor progression or unacceptable toxicities occurred.

As of Oct. 19, 2006, 16 patients (4 in the 10 mg/kg cohort; 3 in each of the other cohorts) had received=1 dose of AMG 655; 12 patients were men, mean (SD) age was 53 (±8.9) years. No DLTs or AMG 655-related serious AEs were reported. The MTD was not reached. Nine patients reported AMG 655-related AEs. Treatment-related AEs in 3 or more patients were: pyrexia (4 patients), fatigue (3 patients), and hypomagnesaemia (3 patients). Fatigue and elevated serum lipase were the only grade 3 or higher AMG 655-related AEs and both occurred in the same patient (0.3-mg/kg cohort). No anti-AMG 655 antibodies were detected. PK data were available from dose cohorts 1 to 3 (0.3, 1, and 3 mg/kg); AMG 655 demonstrated dose-linear kinetics with a half-life of ˜10 days. Tumor-response data were available for 13 patients. Partial response was observed in 1 patient with non-small cell lung cancer (NSCLC) who experienced a 46% reduction in tumor volume by RECIST and remains on study after 48 weeks. Stable disease was reported in 4 patients (range 6 to 35 weeks), and progressive disease in 8 patients. One patient with colorectal cancer and stable disease demonstrated a metabolic partial response with a 34% reduction in maximum standardized uptake value (SUVₘₐₓ).

AMG 655 administered up to 20 mg/kg Q2W appeared to be well tolerated in these patients. The anti-tumor activity of AMG 655 was confirmed with observation of a partial response in NSCLC and a metabolic partial response in colorectal cancer.

Example 2 describes the treatment of patients with metastatic pancreatic cancer with AMG 655 in combination with gemcitabine.

Conatumumab (AMG 655) is an investigational, fully human agonist monoclonal antibody (IgG1) that binds human death receptor 5 (DR5), activates caspases, and induces apoptosis in sensitive tumor cells. In a multi-center phase I trial to evaluate AMG 655 +gemcitabine in metastatic pancreatic patients. The primary endpoint was dose-limiting toxicity (DLT). Secondary endpoints included toxicity, pharmacokinetics, antibody formation, objective response rate, progression-free survival (PFS), 6-month and overall survival.

Eligible patients had previously untreated metastatic pancreatic cancer and ECOG PS (Eastern Cooperative Oncology Group Performance Status) 0 or 1. Patients were enrolled into sequential cohorts and received AMG 655 3 or 10 mg/kg IV days (D) 1 and 15 and gemcitabine 1000 mg/m²IV D 1, 8, and 15 every 28 D. CT scans were obtained Q2 cycles.

Thirteen patients (3 mg/kg cohort=6; 10 mg/kg cohort=7) enrolled from July 2007-November 2007. Patient characteristics: females 61%; ECOG PS 0: 31%, PS 1 69%; median age 65 (range 35-81); liver metastases 77%. Median number of cycles: 6 (range 2-12). There were no DLT. Nine (69%) patients had grade 3-4 toxicity, the most common being thrombocytopenia (4 patients), neutropenia (2 patients), and abdominal pain (2 patients). No anti-AMG 655 antibodies were detected. After one 3- or 10-mg/kg dose of AMG 655 after gemcitabine, the Cₘₐₓ and AUC (area under the curve) of AMG 655 were similar to those in the first-in-human single-agent study (LoRusso JCO 2007; 25: abstract 3534). Preliminary data indicate no effect of AMG 655 on pharmacokinetic (PK) of gemcitabine.

Partial response 4 (31%, 2 unconfirmed), stable disease 5 (38%). Median progression-free survival 5.3 months (95% CI (confidence interval), 3.5, 6.2); 6-month survival rate 76.2% (95% CI: 42.7%-91.7%). Disease control rate (partial responders+stable disease): 69%.

Median overall survival: 11.0 months (95% CI: 6.9%-17%) compared to approximately 6 months (50% survival) and 1 year (17% survival) for gemcitabine alone. Moore et al., Journal of Clinical Oncology, May 20, 2007: 1960-1966.

1. A method for inhibiting the growth of adenocarcinoma of the pancreas in a human patient comprising administering to said patient a therapeutically effective amount of a DR5 agonist and gemcitabine.

2. The method of claim 1, wherein said DR5 agonist is an isolated monoclonal antibody.

3. The method of claim 2, wherein said monoclonal antibody is a fully human monoclonal antibody.

4. The method of claim 3, wherein said monoclonal antibody is conatumumab.

5. The method of claim 3, wherein said monoclonal antibody is administered at from about 3 mg/kg to 20 mg/kg and said gemcitabine is administered at from about 800 to 1200 mg/m².

6. The method of claim 5, wherein said antibody is administered at 10 mg/kg and said gemcitabine is administered at 1000 mg/m².

7. The method of claim 6, wherein said monoclonal antibody is administered on days 1 and 15 of a 28-day cycle and said gemcitabine is administered on days 1, 8, and 15 of said 28-day cycle.

8. The method of claim 7, wherein said patient has stage III or stage IV pancreatic cancer.

9. The method of claim 1, wherein said human patient is homozygous or heterozygous for the valine158 polymorphism in FcγRIIIA, wherein said DR5 agonist is an IgG1 Fc-peptide fusion or IgG1 antibody.

10. The method of claim 9, wherein said human patient is homozygous for the valine158 polymorphism in FcγRIIIA.

11. A method for inhibiting the growth of adenocarcinoma of the pancreas in a human patient comprising:
(a) identifying a human patient with an adenocarcinoma of the pancreas wherein said patient is homozygous or heterozygous for the valine158 polymorphism in FcγRIIIA;
(b) administering to said patient a therapeutically effective amount of a DR5 agonist and gemcitabine, wherein said DR5 agonist is an IgG1 antibody or IgG1 Fc-peptide fusion.

12. The method of claim 11, wherein said human patient is homozygous for the valine158 polymorphism of FcγRIIIA.

13. The method of claim 11, wherein said DR5 agonist has a fucosylated Fc.

14. A method for inhibiting the growth of adenocarcinoma of the pancreas in a human patient comprising:
(a) identifying a human patient with an adenocarcinoma of the pancreas wherein said patient is homozygous or heterozygous for the phenylalanine158 polymorphism in FcγRIIIA;
(b) administering to said patient a therapeutically effective amount of a DR5 agonist and gemcitabine, wherein said DR5 agonist is an IgG1 antibody or IgG1 Fc-peptide fusion.

15. The method of claim 14, wherein said human patient is homozygous for the phenylalanine158 polymorphism in FcγRIIIA.

16. The method of claim 14, wherein said DR5 agonist has an afucosylated Fc.

17. The method of claim 14, wherein said DR5 agonist is a fully-human IgG1 antibody and wherein said Fc of said antibody has a substitution at residue 332 numbered according to the EU index of Kabat, wherein said substitution increases the affinity of said Fc to FCGR3A.

18. A method of selecting a human patient with adenocarinoma of the pancreas for treatment with a therapeutically effective amount of a DR5 agonist and gemcitabine, comprising:
(a) identifying the genotype for FcγRIIIA of said patient for the F158 or V158 allele;
(b) selecting a patient that is heterozygous or homozygous for the V158 allele of FcγRIIIA for said treatment.

19. The method of claim 18, wherein said patient is homozygous for said V158 allele.
